(19) United States
(12) Patent Application Publication
(10) Pub. No.: US 2003/0153539 A1
(43) Pub. Date: Aug. 14, 2003

(54) Method to potentiate the therapeutic efficacy of taxane and derivatives thereof

(76) Inventors: Marius Monshouwer, Via Vittorio Alfrieri (IT); Robert Ings, Highland Park, IL (US); Maurizio Rocchetti, Rho (IT)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/182,728

(22) PCT Filed: Feb. 1, 2001
(86) PCT No.: PCT/EP01/01088

(30) Foreign Application Priority Data
Feb. 11, 2000 (GB) 0003201.1

(51) Int. Cl. A61K 31/66; A61K 31/337; A61K 31/56

(52) U.S. Cl. 514/170; 514/449; 514/125

(57) ABSTRACT

The use of estramustine phosphate and its metabolites estramusfine and estromustine allow to potentiate the therapeutic efficacy of taxanes by both improving their pharmacokinetic and pharmacodynamidc profile through the inhibition of (CYP)2C8 and (CYP)3A4 enzymes, both responsible for the metabolism of the taxanes; formulations of estramustine phosphate and metabolites, combinations of these latter with taxanes and therapeutic methods of treatment comprising them as a combined therapy are also disclosed.

The present invention relates to a method for potentiating the therapeutic efficacy of taxanes and, more particularly, to a method for potentiating the therapeutic efficacy of taxanes by improving the pharmacokinetic as well as pharmacodynamic profile of the taxanes themselves.

Among the family of taxanes, widely known as antitumor agents, is taxol (paclitaxel), a natural product derived from the yew tree having a significant clinical activity against a broad range of tumor types such as, for instance, breast, lung, head and neck, bladder and platinum-refractory ovarian carcinoma (Rowinsky, 1997).

When administered to patients, the drug is readily metabolized to several hydroxylated products. All metabolites so far characterized have been found to be less cytotoxic than paclitaxel itself (Harris, 1994; Kumar, 1995).

Substantially analogous metabolic pathway, hence leading to metabolites which retain cytotoxicity, but to a lesser extent, has also been observed for other taxanes such as, for instance, taxotere (docetaxel).

Previous studies in vitro identified that taxanes are extensively metabolized, through hepatic route, by the action of cytochrome P450 enzymes.

Within this family (CYP)2C8 and (CYP)3A4 are the main enzymes which appear to be involved in taxanes metabolism (Cresteil, 1994; Rahman, 1994).

When referring to paclitaxel, in particular, the formation of the metabolite 6α-hydroxypaclitaxel is catalyzed by (CYP)2C8 whereas the other metabolite p-hydroxyphenyl-C3′-paclitaxel is formed by (CYP)3A4.

6α-Hydroxypaclitaxel or, alternatively, p-hydroxyphenyl-C3′-paclitaxel have been observed to be the pre-dominant metabolites (Desai, 1998).

Dihydroxypaclitaxel is then formed by subsequent hydroxylation of p-hydroxyphenyl-C3′-paclitaxel by CYP2C8 and of 6a-hydroxypaclitaxel by CYP3A4. The roles of both enzymes are summarized as set forth in FIG. 1.

Due to the importance of hepatic elimination of taxanes by the cytochrome P450 family, either an induction or inhibition of the cytochrome P450 isoenzymes involved in the metabolism could result in either an increase or decrease of the clearance of these drugs.

As such, the unfavorable pharmacokinetics of taxanes, due to rather rapid hepatic metabolism, results in the need for more frequent and/or higher than desirable doses for these drugs.

Therefore, inhibition of cytochrome P450 mediated taxanes metabolism will improve the pharmacokinetics (i.e. decrease clearance) of these drugs.

Several drugs have been described in the art as inhibitors of cytochrome P450 (iso)enzymes, and concomitant treatment of these drugs proved to alter the pharmacokinetics of drugs whose elimination is cytochrome P450 dependent.

See, as an example, the use of Ritonavir as cytochrome P450 inhibitor as reported in WO 97/01349 in the name of Abbott Laboratories.

In this respect, for therapeutic purposes, any inhibitor of cytochrome P450 mediated taxane metabolism, should be an inhibitor of both (CYP)2C8 and (CYP)3A4. At present, however, no such clinically-usable inhibitors have been reported as yet.

We unexpectedly found that the co-administration of estramustine phosphate (Estracyt®), an estradiol-17-[beta]-phosphate derivative widely used in the treatment of patients with advanced prostate cancer, resulted to be particularly effective in potentiating the therapeutic efficacy of taxanes. The said beneficial therapeutic effects, therefore, allow much lower and/or frequent doses of taxanes to be administered to a patient in need thereof.

Estramustine phosphate is a pro-dug that is converted to two main active metabolites: initially, estramustine phosphate is hydrolyzed to estramustine which, in turn, is metabolized by oxidation to estromustine.

As per the pro-drug itself, we found that also the main metabolites estramustine and estromustine resulted highly effective in inhibiting both (CYP)2C8 and (CYP)3A4 isoenzymes responsible for taxanes metabolism and clearance.

Therefore, it is a first object of the present invention the use of estramustine phosphate or metabolites thereof in the preparation of a medicament which potentiates the therapeutic efficacy of taxanes.

The said therapeutic effect, in particular, is exerted through (CYP)2C8 and (CYP)3A4 enzymes inhibition.

As above reported, the inhibitory activity towards (CYP)2C8 and (CYP)3A4 enzymes, hence leading to an improvement of the pharmacokinetic and pharmacodynamic profile of co-administered taxanes, has been observed with estramustine phosphate itself as well as with its metabolites estramustine and estromustine. Therefore, also the use of these latter in potentiating the therapeutic efficacy of taxanes is within the scope of the invention.

The dosage of estramustine phosphate or metabolites according to the invention, either as a single administration or repeated in a serial manner, will depend upon several factors such as, for instance, the selected schedule treatment comprising the therapy with taxanes.

High doses of estramustine phosphate or metabolites are however preferred.

Estramustine phosphate is a drug already known for both intravenous and oral administration. In this respect, the use of estramustine phosphate or metabolites thereof in potentiating the therapeutic efficacy of taxanes, according to the present invention, can also be accomplished by administering the drug through intravenous or oral route. Of course, as oral estramustine phosphate is rapidly first-pass metabolized into estramustine and estromustine, it is clear to the man skilled in the art that the use of oral estramustine phosphate in inhibiting (CYP)2C8 and (CYP)3A4 enzymes, hence leading to the desired therapeutic effect, is only exerted by the estramustine phosphate metabolites estramustine and estromustine.

On the other side, when referring to an intravenous administration of estramustine phosphate, the above inhibitory activity appears to be exerted by the prodrug estramustine phosphate itself as well as by the two metabolites estramustine and estromustine.

According to a preferred embodiment of the invention, therefore, the use of estramustine phosphate and metabolites thereof is intended for intravenous administration.

In the present description and in any embodiment of the present invention, unless otherwise specified, when referring to estramustine phosphate or metabolites administered through intravenous route, we intend any intravenous infusion given as a bolus, otherwise solely referred to as i.v. push, or as a slow infusion given for a time varying from about 30 minutes to about 3 hours.

According to another preferred embodiment of the invention, any single intravenous infusion of estramustine phosphate or metabolites is intended at high doses, for instance exceeding 1300 mg or 950 mg/m².

Taxanes, whose therapeutic efficacy is potentiated according to the present invention, are those metabolized by cytochrome P450 enzymes such as, for instance, paclitaxel or docetaxel, independently from their administration route, formulation or schedule treatment comprising them.

As an example, the above taxanes can be formulated according to conventional means for intravenous administration or, alternatively, encapsulated within liposomes.

According to a preferred embodiment of the invention, the use of estramustine phosphate or metabolites thereof is intended to potentiate the therapeutic efficacy of paclitaxel.

According to another preferred embodiment of the invention, the use of estramustine phosphate or metabolites thereof is intended to potentiate the therapeutic efficacy of taxotere.

It is a further object of the present invention a formulation of estramustine phosphate or metabolites thereof for use in potentiating the therapeutic efficacy of taxanes by inhibiting (CYP)2C8 and (CYP)3A4 enzymes.

As set forth above, a preferred formulation of the invention comprises estramustine phosphate or metabolites for intravenous use.

The said formulations are used in therapy in the treatment of cancer such as, for instance, prostate cancer, breast cancer, melanoma, lung cancer, pancreatic cancer, colorectal cancer, ovarian cancer and cancers of the brain.

A further object of the present invention is a combination which potentiates the therapeutic efficacy of taxanes by inhibiting (CYP)2CS and (CYP)3A4 enzymes, which combination comprises estramustine phosphate or metabolites thereof for administration on the day of, or within 3 days of, administration of the taxane derivative.

Preferably, the said combination comprises the intravenous administration of estramustine phosphate or metabolites thereof.

Still another object of the invention is a product comprising estramustine phosphate or metabolites thereof and a taxane, as a combined preparation for simultaneous, separate or sequential use in anticancer therapy, wherein the said product is intended for potentiating the efficacy of the above taxane by improving its pharmacokinetic and pharmacodynamic profile.

Pharmaceutically acceptable carriers or excipients to be utilized in the preparation of a medicament or pharmaceutical composition according to the invention are well known to people skilled in the art of formulating compounds in a form of pharmaceutical compositions.

For example, such pharmaceutical compositions may routinely contain, e.g. pharmaceutically acceptable salts, buffering agents, preservatives and/or compatible carriers, especially those used in intravenous formulations. As used herein, “pharmaceutically acceptable carrier” refers to one or more compatible solid or liquid filler, diluent or encapsulating substances which are suitable for administration to mammals including humans.

Pharmaceutical compositions suitable for parenteral administration are typically formulated in a sterile form. The sterile composition thus may be a sterile solution or suspension in a non-toxic parenterally acceptable diluent or solvent.

Optionally, the aforementioned product, combination, formulation or use according to the present invention may further comprise another chemotherapeutic agent such as, for instance, CPT-11, SN-38, camptothecin derivatives, anthracycline glycosides, e.g., doxorubicin, idarubicin, epirubicin, etoposide, navelbine, vinblastine, carboplatin, cisplatin, celecoxib, parecoxib, rofecoxib, valecoxib, JTE 5222, Sugen SU-5416, Sugen SU-6668, Herceptin, and the like, optionally within liposomal formulations thereof.

### BRIEF DESCRIPTION OF FIGURES

FIG. 1: metabolic pathway of paclitaxel biotransformation in human;

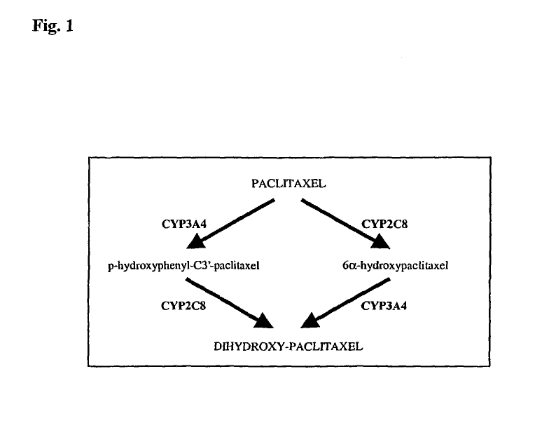

FIG. 2: inhibition of CYP2C8 mediated 6a-hydroxylation of paclitaxel by estromustine and estramustine at 100 μM. Results are presented as percentage of activity remaining in the presence of the compound. Values are mean±SD (N=3).

### PHARMACOLOGY

To study drug-drug interactions with taxanes it is not possible to use rodent models, either in vivo or in vitro, because of the metabolism of taxanes in rats and mice which is qualitatively and quantitatively different from that in humans (Monsarrat, 1993; Eiseman, 1994; Sparreboom, 1996).

Thus, for a taxane drug such as, for instance, paclitaxel, the need for carrying out in vitro studies using human derived material is clearly evident.

The key rationale for performing in vitro experiments on drug-drug interactions is the presumed applicability to the clinical situation. At present there is a growing body of literature that indicates that in many cases, in vitro studies are predictive of results in vivo (Hoener, 1994; Houston, 1994).

Although in vitro studies suggested that paclitaxel is mainly metabolized by CYP2C8 and to a lesser extent by CYP3A4 (Cresteil, 1994; Rahman, 1994) it has been recently shown that the role of CYP3A4 in vivo is as important as of CYP2C8 (Monsarrat, 1998).

The apparent discrepancy between the in vitro and in vivo observation might be due to induction of CYP3A4. Cancer patient receiving paclitaxel, in fact, are commonly pretreated with corticosteroids, known as inducers of CYP3A4 enzymes.

Whether in vitro inhibition of the two enzymes involved in taxanes metabolism, CYP3A4 and CYP2C8, is relevant for in vivo situation, highly depends on the plasma concentration levels of estramustine phosphate, estramustine and estromustine.

Nowadays, high estramustine phosphate doses are considered in advanced cancer therapy. For instance, doses of estramustine phosphate up to 2000 mg/m² result in plasma levels of both estramustine and estromustine well above 10 μM. As both estramustine phosphate and the two major metabolites, estramustine and estromustine appear to inhibit cytochrome P450, it should be realized the importance of the sum of the plasma levels of these three compounds.

As previously indicated, therefore, since both CYP3A4 and CYP2C8 enzymes play a primary role in the disposition of taxanes, inhibition of both CYP3A4 and CYP2C8 mediated enzyme activities by estramustine phosphate or metabolites thereof, preferably at high doses, would result in a decreased hepatic metabolism of taxanes and, as a consequence, a decreased excretion.

With the aim of better illustrating the present invention, without posing any limitation to it, herewith reported are some examples showing the inhibitory activity towards specific cytochrome P450 enzymes of estramustine phosphate, estramustine and estromustine.

### EXAMPLE 1

Chemicals

¹⁴C-Chlorzoxazone, [phenylacetic acid ring-U-1⁴C]diclofenac sodium, S-[4¹⁴C] mephenytoin, [4-¹⁴C]testosterone, were purchased from Amersham Pharmacia Biotech (Buckinghamshire, UK). and ¹⁴C-Delavirdine mesylate (PNU-90152E) was obtained from Pharmacia & Upjohn, Kalamazoo, Mich., USA. Human CYP1A2, CYP2C9, CYP2C19, CYP2D6, and CYP3A4, were purchased from Gentest (Woburn, Mass., USA). Estramustine phosphate, estramustine and estromustine were either commercially available or, alternatively, prepared according to well known methods.

Other reagents and solvents were analytical grade and were commercially available.

Incubation

The ability of estramustine phosphate, estramustine and estromustine to inhibit P450 enzymes was investigated in vitro against five different cDNA expressed human cytochrome P450 (CYP) enzyme systems (CYP1A2, CYP2C9, CYP2C19, CYP2D6, and CYP3A4). CYP enzyme was used in an amount giving a turnover of the marker substrate of 10-20%, while the substrate concentration corresponded to its Km value (see table 1).

TABLE 1
Incubation conditions for in vitro inhibition assay

| | | | | Incubation time |
|---|---|---|---|---|
| Enzyme | Pmol/well | Substrate | μM | (min) |
| CYP1A2 | 2 | Chlorzoxazone | 8.5 | 30 |
| CYP2C9 | 0.35 | Diclofenac | 10 | 90 |
| CYP2C19 | 3 | (S)-mephenytoin | 20 | 90 |
| CYP2D6 | 0.7 | Delavirdine | 10 | 90 |
| CYP3A4 | 0.65 | Testosterone | 50 | 90 |

Estramustine phosphate was dissolved in 0.1 M KH₂PO₄ (pH 7.4), whereas estromustine and estramustine were dissolved in DMSO:CH₃CN (1:1). The reaction, in a final volume of incubation of 100 IA, was started by adding NADPH. After 90 min incubation (30 min for CYP1A2) at 37° C., the reaction was stopped by adding 50 μl CH₃CN followed by additional 50 μl of mobile phase. At the end, samples were centrifuged at 1200 g for 15 min at 4° C. and radio-HPLC analyzed. All incubations were conducted in triplicate.

Radio-HPLC Analyses

Quantitation of substrates and their metabolites were achieved using an HPLC system equipped with a Radiomatic Flo-One radioactivity flow detector (Packard). Analytical separations of substrates and metabolites were performed on a Zorbax SB-C8 column, 4.6×150 mm, 5 μm (Hewlett Packard, Waldbronn, Germany), plus a 3Nucleosil 120-3 C18, 4×30 mm precolumn (Macherey-Nagel, Duren, Germany). For testosterone, analytical separations of substrate and metabolite were performed on a Zorbax SB-C18 column, 4.6×150 mm, 5 μm (Hewlett Packard), plus a 3Nucleosil 120-3 C18, 4×30 mm precolumn (Macherey-Nagel). See table 2 for HPLC mobile phase conditions.

TABLE 2
Mobile phase and flow conditions for the radio-HPLC analyses

| | | | Flow |
|---|---|---|---|
| Assay | A (%) | B (%) | (ml/min) |
| | Mobile phase A | | |
| | [H₂O:CH₃OH:CH₃COOH = 90:10:0.2] | | |
| | Mobile phase B | | |
| | [CH₃OH:H₂O:CH₃COOH = 90:10:0.2] | | |
| Chlorzoxazone | 40 | 60 | 1 |
| (S)-mephenytoin | 40 | 60 | 1 |
| Delavirdine | 30 | 70 | 1 |
| Diclofenac | | | 1.5 |
| Time (min) | | | |
| 0 | 80 | 20 | |
| 7 | 10 | 90 | |
| 10 | 10 | 90 | |
| 11 | 80 | 20 | |
| 13 | 80 | 20 | |
| | Mobile phase A | | |
| | [CH₃CN:H₂O:CH₃COOH = 20:80:0.25] | | |
| | mobile phase B | | |
| | [CH₃CN:H₂O:CH₃COOH = 65:35:0.25] | | |
| Testosterone | 20 | 80 | 1 |

The resulting inhibitory effect exerted by estramustine phosphate, estramustine and estromustine against several human CYP mediated enzyme activities is presented in table 3.

TABLE 3
Inhibition of major human cytochrome P450 enzymes by estramustine
phosphate, estromustine, and estramustine at either 10 or 100 μM.
Results are presented as percentage of activity remaining in the
presence of the compound. Values are mean ± SD (n = 3).
Control (absence of the compound) values are 100%.

| | Estramustine | | | | | |
|---|---|---|---|---|---|---|
| | phosphate | | Estromustine | | Estramustine | |
| CYP | 10 μM | 100 μM | 10 μM | 100 μM | 100 μM | 100 μM |
| CYP1A2 | 99.8 ± | 61.3 ± | 85.0 ± | 68.4 ± | 72.7 ± | 57.2 ± |
| | 22.8 | 11.9 | 4.9 | 8.0 | 11.4 | 17.3 |
| CYP2C9 | 88.6 ± | 71.6 ± | 92.5 ± | 86.9 ± | 56.4 ± | 58.8 ± |
| | 17.3 | 10.2 | 3.1 | 7.2 | 1.4 | 3.8 |
| CYP2C19 | 93.9 ± | 87.5 ± | 90.6 ± | 81.0 ± | 37.9 ± | 40.1 ± |
| | 2.6 | 4.5 | 2.1 | 9.1 | 10.0 | 5.0 |
| CYP2D6 | 87.1 ± | 70.7 ± | 64.1 ± | 76.9 ± | 38.0 ± | 32.7 ± |
| | 0.9 | 7.0 | 19.9 | 2.81 | 5.6 | 25.5 |
| CYP3A4 | 80.7 ± | 47.9 ± | 43.2 ± | 33.1 ± | 27.0 ± | 20.2 ± |
| | 2.5 | 4.8 | 4.8 | 1.6 | 2.3 | 4.3 |

From the above table clearly appears that at a concentration of 100 μM, all the compounds were able to inhibit CYP activities.

In particular, 60-hydroxylation of testosterone (CYP3A4) was affected and an inhibition of approximately 80% was observed with estramustine. Of all the three compounds, estramustine resulted to be the most potent inhibitor.

### EXAMPLE 2

Chemicals

Paclitaxel was obtained from Sigma. Human CYP2C8, was purchased from Gentest (Woburn, Mass., USA). Estramustine and estromustine Pharmacia & Upjohn (Nerviano, Italy). Other reagents and solvents were analytical grade and were commerical available.

Incubations

A 100 μl reaction mixture containing 4 pmol CYP2C8, 20 PM paclitaxel (5 mM paclitaxel stock in ethanol) and NADPH (2 mM) in 50 mM potassium phosphate buffer (pH 7.4) was incubated at 37° C. for 40 min in the absence (solvent only) and presence of either 100 μM estramustine or estromustine.

Estramustine and estromustine were dissolved in DMSO:CH₃CN (1:1). The reaction was stopped by addition of 50 μl CH₃CN followed by an additional 50 μl of mobile phase. Finally samples were centrifuged at 1200 g for 15 min at 4° C. and analyzed radio-HPLC. All incubations were conducted in triplicate.

HPLC Analyses

Quantitation of substrates and their metabolites were achieved using an HPLC system equipped with a UV detector. 125 μl of the supernatent was injected into a Zorbax SB-C18 column, 4.6×150 mm, 5 μm (Hewlett Packard, Waldbronn, Germany), and separated at 45° C. with a mobile phase initially of 58% methanol increasing to 82% methanol over 20 min and at a flow rate of 1.0 ml/min. See table 2 for HPLC mobile phase conditions. Both paclitaxel and the metabolite 6a-hydroxypaclitaxel were detected by its absorbance at 230 nm.

There is currently limited information regarding inhibitors, inducers and substrates of CYP2CS in man, in vivo. Only a few clinically utilized drugs, for example paclitaxel and cerivastatin, are known to be substrates for CYP2C8 (Sonnichsen,1995; Wolfgang, 1998).

To investigate if estramustine and estromustine were able to inhibit CYP2C8 mediated enzyme activities, human CYP2C8 cDNA expressed microsomes were incubated with paclitaxel in the presence of either estramustine or estromustine.

In particular, at a concentration of 100 μM, estromustine or estramustine were able to inhibit 6α-hydroxylation of paclitaxel by 20% and 40%, respectively.

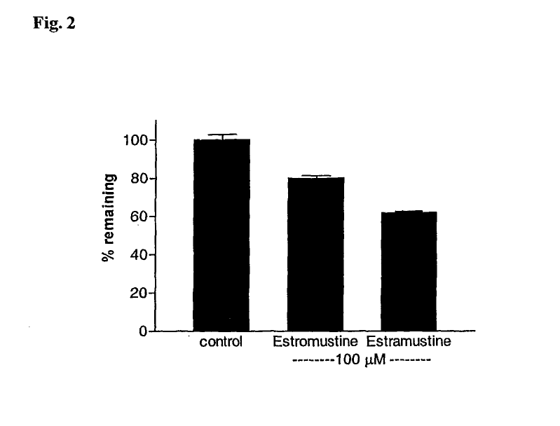

The results, expressed as percentage of activity remaining in the presence of the tested compound [values are mean±SD (n=3)], are reported in FIG. 2.

### REFERENCES

Cresteil T, Monsarrat B, Alvinerie P, Treluyer J M, Vieira I, Wright M. Taxol metabolism by human liver microsomes: identification of cytochrome P450 isozymes involved in its biotrans-formation. Cancer Res 1994;54:386-92.

Desai P B. Duan J Z. Zhu Y W. Kouzi S. Human liver microsomal metabolism of paclitaxel and drug interactions. Eur J Dnig Metabol Pharmacokin 1998;23:417-24.

Eiseman J L, Eddington N D, Leslie J, et al. Plasma pharmacokinetics and tissue distribution of paclitaxel in CD2F1 mice. Cancer Chemotlier Pharmacol 1994;34:465-71.

Harris J W, Katki A, Anderson L W, Chmurny G N, Paukstelis J V, Collins J M. Isolation, structural determination, and biological activity of 6 alpha-hydroxy-taxol, the principal human metabolite of taxol. J Med Chem 1994;37:706-9.

Hoener B A. Predicting the hepatic clearance of xenobiotics in humans from in vitro data. Biopharm Drug Dispos 1994;15:295-304.

Houston J B. Utility of in vitro drug metabolism data in predicting in vivo metabolic clearance. Biochem Pharmacol 1994;47: 1469-79.

Kumar G N, Ray S, Walle T, Huang Y, Willingham M, Self S and Bhalla K N. Comparative in vitro cytotoxic effects of taxol and its major human metabolite 6α-hydroxytaxol. Cancer Chemother Pharmcol 1995;36: 129-135.

MonsarTat B, Alvinerie P, Wright M, et al. Hepatic metabolism and biliary excretion of Taxol in rats and humans. J Natl Cancer Inst Monogr 1993;15:39-46.

Monsarrat B. Chatelut E. Royer I. Alvinerie P. Dubois J. Dezeuse A. Roche H. Cros S. Wright M. Canal P. Modification of paclitaxel metabolism in a cancer patient by induction of cytochrome P450 3A4. Drug Metabol Disp 1998;26:229-233.

Rahman A, Korzekwa K R, Grogan J, et al. Selective biotransformation of taxol to 6 alpha-hydroxytaxol by human cytochrome P450 2C8. Cancer Res 1994;54:5543-6.

Rowinsky E K. The development and clinical utility of the taxane class of antimicrotubule chemotherapy agents. Ann Rev Med 1997;48:353-374.

Sonnichsen D S, Liu Q, Schuetz E G, Schuetz J D, Pappo A, Relling M V. Variability in human cytochrome P450 paclitaxel metabolism. J Phamacol Exp Ther 1995; 275: 566-575.

Sparreboom A, van Tellingen O, Nooijen W J, et al. Tissue distribution, metabolism and excretion of paclitaxel in mice. Anticancer Drugs 1996;7:78-86.

Wolfgang M. Rational assessment of the interaction profile of cerivastatin supports its low propensity for drug interactions. Drugs 1998; 56:15-23.

1. Use of estramustine phosphate or a metabolite thereof in the preparation of a medicament which potentiates the therapeutic efficacy of a taxane.

2. Use according to claim 1 wherein the therapeutic efficacy of the taxane is potentiated by inhibiting (CYP)2C8 and (CYP)3A4 enzymes.

3. Use according to claim 1 wherein the medicament is formulated for the administration of estramustine phosphate or a metabolite thereof at a high dose.

4. Use according to claim 1 wherein the medicament is for intravenous administration.

5. Use according to claim 3 wherein the high dose of estramustine phosphate or a metabolite thereof is in excess of 1300 mg or 950 mg/m² for a single intravenous infusion.

6. Use according to claim 1 wherein the estramustine phosphate metabolite is estramustine or estromustine.

7. Use according to claim 1 wherein the taxane is selected from paclitaxel and docetaxel, both optionally encapsulated within liposomes.

8. Use according to claim 1 wherein the taxane is paclitaxel.

9. An agent for use in potentiating the therapeutic efficacy of a taxane comprising estramustine phosphate or a metabolite thereof.

10. An agent according to claim 9 wherein the therapeutic efficacy of the taxane is potentiated by inhibiting (CYP)2C8 and (CYP)3A4 enzymes.

11. An agent according to claim 9 comprising a high dose of estramustine phosphate or a metabolite thereof.

12. An agent according to claim 9 for intravenous use.

13. An agent according to claim 12 formulated to deliver a single intravenous infusion dosage of estramustine phosphate or a metabolite thereof in excess of 1300 mg or 950 mg/m².

14. An agent according to claim 9 wherein the estramustine phosphate metabolite is estramustine or estromustine.

15. An agent according to claim 9 wherein the taxane is selected from paclitaxel and docetaxel, both optionally encapsulated within liposomes.

16. An agent according to claim 9 wherein the taxane is paclitaxel.

17. An agent according to claim 9 for use in the treatment of cancer.

18. An agent according to claim 17 wherein the cancer is selected from prostate cancer, breast cancer, melanoma, lung cancer, pancreatic cancer, colorectal cancer, ovarian cancer and cancers of the brain.

19. An agent according to claim 9 which is in the form of a pharmaceutical composition which further includes a pharmaceutically acceptable carrier or diluent.

20. A combination which potentiates the therapeutic efficacy of a taxane which comprises estramustine phosphate or a metabolite thereof for administration on the day of, or within 3 days of, administration of the said taxane.

21. A combination according to claim 20 wherein the therapeutic efficacy of the taxane is potentiated by inhibiting (CYP)2C8 and (CYP)3A4 enzymes.

22. A combination according to claim 20 wherein estramustine phosphate or the metabolite thereof is formulated for administration at a high dose.

23. A combination according to claim 20 wherein estramustine phosphate or the metabolite thereof is formulated for intravenous administration.

24. A combination according to claim 23 wherein the dosage of estramustine phosphate or a metabolite thereof is in excess of 1300 mg or 950 mg/m² for a single infusion.

25. A combination according to claim 20 wherein the estramustine phosphate metabolite is estramustine or estromustine.

26. A combination according to claim 20 wherein the taxane is selected from paclitaxel and docetaxel, both optionally encapsulated within liposomes.

27. A combination according to claim 20 wherein the taxane is paclitaxel.

28. A combination according to claim 20 for use in the treatment of cancer.

29. A combination according to claim 28 wherein the cancer is selected from prostate cancer, breast cancer, melanoma, lung cancer, pancreatic cancer, colorectal cancer, ovarian cancer and cancers of the brain.

30. A product comprising estramustine phosphate or a metabolite thereof and a taxane as a combined preparation for simultaneous, separate or sequential use in anticancer therapy.

31. A product according to claim 30 wherein the said estramustine phosphate or metabolite thereof serves to potentiate the therapeutic efficacy of the said taxane.

32. A product according to claim 31 wherein the therapeutic efficacy of the taxane is potentiated by inhibiting (CYP)2C8 and (CYP)3A4 enzymes.

33. A product according to claim 30 comprising an additional chemotherapeutic agent.

34. A product according to claim 33 wherein the additional chemotherapeutic agent is selected from CPT-11, doxorubicin, etoposide, navelbine, vinblastine carboplatin and cisplatin.

35. A product according to claim 30 wherein the estramustine phosphate or metabolite thereof is formulated for intravenous use.

36. A product according to claim 35 wherein the dosage of estramustine phosphate or the metabolite thereof is in excess of 1300 mg or 950 Mg/M2 per single infusion.

37. A product according to claim 30 wherein the estramustine phosphate metabolite is estramustine or estromustine.

38. A product according to claim 30 wherein the taxane is selected from paclitaxel and docetaxel, both optionally encapsulated within liposomes.

39. A product according to claim 30 wherein the taxane is paclitaxel.

40. A method of treating a patient in need of taxane therapy, which method comprises administering estramustine phosphate or a metabolite thereof and a taxane to the said patient.

41. A method of potentiating the therapeutic efficacy of a taxane, which method comprises administering to a patient in need thereof a taxane and estramustine phosphate or a metabolite thereof.

42. A method according to claim 40 or 41 wherein the taxane and the estramustine phosphate or metabolite thereof are administered simultaneously, separately or sequentially to the said patient.

43. A method according to claim 40 or 41 wherein the estramustine phosphate or metabolite thereof and the taxane are administered as a combined preparation for simultaneous, separate or sequential use.

44. A method according to claim 40 or 41 wherein estramustine phosphate or the metabolite thereof is administered intravenously on the day of, or within 3 days of, administration of the taxane.

45. A method according to claim 40 or 41 wherein the estramustine phosphate metabolite is estramustine or estromustine.

46. A method according to claim 40 or 41 wherein the taxane is selected from paclitaxel and docetaxel, both optionally encapsulated within liposomes.

47. A method according to claim 40 or 41 wherein the taxane is paclitaxel.

48. Use of estramustine phosphate or a metabolite thereof in the preparation of a medicament which modifies the systemic exposure or the pharmacokinetic profile of a taxane.
